(19) United States
(12) Patent Application Publication
(10) Pub. No.: US 2025/0302961 A1
(43) Pub. Date: Oct. 2, 2025

(54) TARGETING MYELOID-DERIVED SUPPRESSOR CELLS (MDSCS) IN BLADDER CANCER TO ENHANCE EFFICACY OF ADOPTIVE CELL THERAPY (ACT)

(71) Applicant: H. LEE MOFFITT CANCER CENTER AND RESEARCH INSTITUTE, INC., Tampa, FL (US)

(72) Inventors: Matthew BEATTY, Riverview, FL (US); Shari PILON-THOMAS, Land O Lakes, FL (US); Sarah BAZARGAN, Ormond Beach, FL (US); Michael POCH, Tampa, FL (US); Brittany BUNCH, Tampa, FL (US)

(21) Appl. No.: 18/864,634

(22) PCT Filed: May 10, 2023
(86) PCT No.: PCT/US2023/021665

§ 371 Date: Nov. 11, 2024

Related U.S. Application Data
(60) Provisional application No. 63/340,094, filed on May 10, 2022.

(51) Int. Cl. A61K 40/42; A61K 31/513; A61K 31/52; A61K 31/522; A61K 31/7068; A61K 31/7072; A61K 31/7076; A61K 31/708; A61K 40/11

(52) U.S. Cl. CPC A61K 40/428; A61K 31/513; A61K 31/52; A61K 31/522; A61K 31/7068; A61K 31/7072; A61K 31/7076; A61K 31/708; A61K 40/11

(57) ABSTRACT

Disclosed are methods for reducing the immunosuppressive effect of myelin derived suppressor cells in adoptive cell therapy.

### II. CROSS REFERENCE TO RELATED APPLICATIONS

This Application claims the benefit of U.S. Provisional Application No. 63/340,094, filed on May 10, 2022, which is incorporated herein by reference in its entirety.

### I. GOVERNMENT SUPPORT

This invention was made with government support under Grant No. 1R01CA259387 awarded by the National Institutes of Health. The government has certain rights in the invention.

### III. BACKGROUND

MDSCs are a significant barrier to adoptive cell therapy (ACT) due to suppressive effects on T-cells. What are needed are adoptive cell therapy methods that can reduce the suppressive effects of MDSCs.

### IV. SUMMARY

Disclosed are methods for reducing the immunosuppressive effect of myelin derived suppressor cells in adoptive cell therapy.

In one aspect, disclosed herein are methods of treating, decreasing, reducing, inhibiting, ameliorating, and/or preventing a cancer and/or metastasis (such as, for example, bladder cancer) in a subject comprising administering to the subject a therapeutically effective amount of a nucleoside analog (such as, for example, gemcitabine, cytarabine, emtricitabine, lamivudine, zalcitabine, didanosine, vidarabine, abacavir, acyclovir, entecavir,.stavudine, telbivudine, zidovudine, idoxuridine, or trifluridine) followed by the administration of a therapeutically effective amount of an adoptive cell therapy (ACT) (such as for example, administering to the subject a therapeutically effective amount of tumor reactive immune cells (such as, for example, T cells, natural killer (NK) cells, and/or NK T cells including, but not limited to chimeric antigen receptor (CAR) T cells, CAR natural killer (NK) (CAR NK) cells, tumor infiltrating lymphocytes, and/or marrow infiltrating lymphocytes)).

Also disclosed herein are methods of reducing the suppressive effects of myelin derived suppressor cells (MDSCs) in a subject receiving adoptive cell therapy for the treatment, inhibition, reducing, decrease, amelioration and/or prevention of a cancer (such as, for example, bladder cancer) and/or increasing the efficacy of adoptive cell therapy (ACT) for the treatment, inhibition, reducing, decrease, amelioration and/or prevention of a cancer (such as, for example, bladder cancer) comprising administering to the subject a therapeutically effective amount of a nucleoside analog (such as, for example, gemcitabine, cytarabine, emtricitabine, lamivudine, zalcitabine, didanosine, vidarabine, abacavir, acyclovir, entecavir,.stavudine, telbivudine, zidovudine, idoxuridine, or trifluridine) prior to administration of the adoptive cell therapy (such as for example, administering to the subject a therapeutically effective amount of tumor reactive immune cells (such as, for example, T cells, natural killer (NK) cells, and/or NK T cells including, but not limited to chimeric antigen receptor (CAR) T cells, CAR natural killer (NK) (CAR NK) cells, tumor infiltrating lymphocytes, and/or marrow infiltrating lymphocytes)).

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows that bladder tumors are enriched for myelin derived suppressor cells (MDSCs).

FIG. 2 shows that the highest proportion of cells within urine are MDSCs

FIG. 3 shows that MDSCs that infiltrate the tumor are suppressive.

FIG. 4 shows that pretreatment with Gemcitabine in large tumors improves adoptive cell therapy (ACT)

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF TREATING CANCER

We predict that there is an enrichment of MDSCs within bladder cancer and depletion of MDSCs may augment anti-tumor responses after ACT with tumor reactive T-cells.

In one aspect, disclosed herein are methods of treating, decreasing, reducing, inhibiting, ameliorating, and/or preventing a cancer and/or metastasis (such as, for example, bladder cancer) in a subject comprising administering to the subject a therapeutically effective amount of a nucleoside analog followed by the administration of a therapeutically effective amount of an adoptive cell therapy (ACT).

The nucleoside analog used in the disclosed methods can be any nucleoside analaog known in the art, including, but not limited to gemcitabine, cytarabine, emtricitabine, lamivudine, zalcitabine, didanosine, vidarabine, abacavir, acyclovir, entecavir, stavudine, telbivudine, zidovudine, idoxuridine, or trifluridine. Thus, in one aspect, disclosed herein are methods of treating, decreasing, reducing, inhibiting, ameliorating, and/or preventing a cancer and/or metastasis (such as, for example, bladder cancer) in a subject comprising administering to the subject a therapeutically effective amount of gemcitabine, followed by the administration of a therapeutically effective amount of an ACT. In one aspect, ACT can comprise administration of a donor immune cell (including, but not limited to autologous, allogeneic, and/or syngeneic immune cells). The immune cell can be a T cell, natural killer (NK) cells, and/or NK T cell. In one aspect, the immune cell can be obtained from a tumor microenvironment such as tumor infiltrating lymphocytes (TILs), and marrow infiltrating lymphocytes (MILs). In some aspects, the immune cells can be modified such as chimeric antigen receptor (CAR) T cells, CAR natural killer (NK) (CAR NK) cells. Accordingly, in one aspect disclosed herein are methods of treating, decreasing, reducing, inhibiting, ameliorating, and/or preventing a cancer and/or metastasis (such as, for example, bladder cancer) in a subject comprising administering to the subject a therapeutically effective amount of gemcitabine, followed by the administration of a therapeutically effective amount of an ACT (such as, for example, tumor infiltrating lymphocytes.

Administration of the nucleoside analog can occur prior to administration of ACT to the recipient subject. For example, the nucleoside analog can be administered at least 1, 2, 3, 4, 5, 6, 7, 8, 9, 10, 11, 12, 13, 14, 15, 16, 17, 18, 19, 20, 21, 22, 23, 24, 36, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 56, or 60 days before administration of the ACT. In one aspect, disclosed herein are methods of treating, decreasing, reducing, inhibiting, ameliorating, and/or preventing a cancer and/or metastasis (such as, for example, bladder cancer) in a subject wherein the nucleoside analog is administered 7 days prior to administration of the ACT.

While it is preferable for only a single dose of the nucleoside analog to be effective, it is understood and herein contemplated that multiple doses of the nucleoside analog could be required. In one aspect, the subject receives administration of the nucleoside analog 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 56, or 60 times. In some aspects, the nucleoside analog can be administered every 1, 2, 3, 4, 5, 6, 12, 18, 24, 48, 72, 96, hours.

Also disclosed herein are methods of reducing the suppressive effects of myelin derived suppressor cells (MDSCs) in a subject receiving adoptive cell therapy for the treatment, inhibition, reducing, decrease, amelioration and/or prevention of a cancer (such as, for example, bladder cancer) and/or increasing the efficacy of adoptive cell therapy (ACT) for the treatment, inhibition, reducing, decrease, amelioration and/or prevention of a cancer (such as, for example, bladder cancer), said methods comprising administering to the subject a therapeutically effective amount of a nucleoside analog prior to administration of the adoptive cell therapy.

The nucleoside analog used in the disclosed methods can be any nucleoside analaog known in the art, including, but not limited to gemcitabine, cytarabine, emtricitabine, lamivudine, zalcitabine, didanosine, vidarabine, abacavir, acyclovir, entecavir, stavudine, telbivudine, zidovudine, idoxuridine, or trifluridine. Thus, in one aspect, disclosed herein are methods of reducing the suppressive effects of myelin derived suppressor cells (MDSCs) in a subject receiving adoptive cell therapy for the treatment, inhibition, reducing, decrease, amelioration and/or prevention of a cancer (such as, for example, bladder cancer) and/or increasing the efficacy of adoptive cell therapy (ACT) for the treatment, inhibition, reducing, decrease, amelioration and/or prevention of a cancer (such as, for example, bladder cancer) comprising administering to the subject a therapeutically effective amount of gemcitabine, followed by the administration of a therapeutically effective amount of an ACT. In one aspect, ACT can comprise administration of a donor immune cell (including, but not limited to autologous, allogeneic, and/or syngeneic immune cells). The immune cell can be a T cell, natural killer (NK) cells, and/or NK T cell. In one aspect, the immune cell can be obtained from a tumor microenvironment such as tumor infiltrating lymphocytes (TILs), and marrow infiltrating lymphocytes (MILs). In some aspects, the immune cells can be modified such as chimeric antigen receptor (CAR) T cells, CAR natural killer (NK) (CAR NK) cells. Accordingly, in one aspect disclosed herein are methods of reducing the suppressive effects of myelin derived suppressor cells (MDSCs) in a subject receiving adoptive cell therapy for the treatment, inhibition, reducing, decrease, amelioration and/or prevention of a cancer (such as, for example, bladder cancer) and/or increasing the efficacy of adoptive cell therapy (ACT) for the treatment, inhibition, reducing, decrease, amelioration and/or prevention of a cancer (such as, for example, bladder cancer) comprising administering to the subject a therapeutically effective amount of gemcitabine, followed by the administration of a therapeutically effective amount of an ACT (such as, for example, tumor infiltrating lymphocytes.

Administration of the nucleoside analog can occur prior to administration of ACT to the recipient subject. For example, the nucleoside analog can be administered at least 1, 2, 3, 4, 5, 6, 7, 8, 9, 10, 11, 12, 13, 14, 15, 16, 17, 18, 19, 20, 21, 22, 23, 24, 36, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 56, or 60 days before administration of the ACT. In one aspect, disclosed herein are methods of reducing the suppressive effects of myelin derived suppressor cells (MDSCs) in a subject receiving adoptive cell therapy for the treatment, inhibition, reducing, decrease, amelioration and/or prevention of a cancer (such as, for example, bladder cancer) and/or increasing the efficacy of adoptive cell therapy (ACT) for the treatment, inhibition, reducing, decrease, amelioration and/or prevention of a cancer (such as, for example, bladder cancer) wherein the nucleoside analog is administered 7 days prior to administration of the ACT.

While it is preferable for only a single dose of the nucleoside analog to be effective, it is understood and herein contemplated that multiple doses of the nucleoside analog could be required. In one aspect, the subject receives administration of the nucleoside analog 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 56, or 60 times. In some aspects, the nucleoside analog can be administered every 1, 2, 3, 4, 5, 6, 12, 18, 24, 48, 72, 96, hours.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human,,Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo. (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B-and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Methods

For murine studies, orthotopic MB49-OVA bladder tumors were collected, stained for MDSCs, and analyzed by flow cytometry. Urine samples from bladder cancer patients were also collected and stained for MDSCs. From mice, purified MDSCs and OT-I T-cells were cocultured and from bladder cancer patients, purified urine MDSCs and CD3-stimulated peripheral blood T-cells were cocultured to assess suppression of T-cell proliferation or IFN-gamma secretion. Mice bearing MB49-OVA tumors were treated with intravesical instillation of gemcitabine and/or OT-I T-cells and tumor growth was monitored via ultrasound.

### (1) MB49-OVA Bladder Cancer Model:

C57BL/6 female mice were initially anesthetized and catheterized (Terumo), then pre-treated orthotopically with Poly-L-Lysine in PBS (50 uL at 1μg/mL) for 10 minutes. Then bladders were washed with 50 uL PBS and mice received intravesical instillation of 1×10⁵ MB49-OVA murine bladder cancer cells for 30 minutes. Presence of bladder tumors were confirmed via the Vevo 2100 ultrasound system (FUJIFILM VisualSonics Inc.) 10 days after tumor inoculation and ultrasound was performed 1X per week until mice were euthanized.

### (2) Flow Cytometry:

For murine experiments, tumors were harvested and processed into single-cell suspension via enzymatic and mechanical digestion and treated with red blood cell (RBC) lysis buffer to remove red blood cells. Cell were stained with Live/Dead viability dye then with two panels: the first panel staining for the markers CD4, CD8, Nk1.1, Ova-tetramer, and CD45 and the second panel staining for the markers Ly6C, MHCII, CD45, F4/80, CD11b, CD19, CD103, Ly6G, and CD11c. For analysis of patient urine, samples were centrifuged and stained with Live/Dead viability dye, HLA-DR, CD3, CD19, CD56, CD14, CD11b, CD33, LOX1, and CD15. All samples were run on FACSCelesta flow cytometer and analyzed using FlowJo V10 software.

### (3) MDSC Suppression Assays

To evaluate the suppressive effects of MDSCs in bladder cancer, spleens were collected from MB49-OVA-bearing mice. MDSCs from splenocytes were isolated by positively selecting for Gr-1+ cells. T cells were isolated from OT-I TCR transgenic mice and labeled with CellTrace Violet (CTV). T cells were unstimulated, stimulated with OVA peptide alone or in combination with purified MDSCs for 66-hours. Proliferation of T cells was analyzed by flow cytometry and 1:1 MDSC to OT-I T-cell culture resulted in a dilution of CTV and a reduction in the number of CTV peaks. To determine whether MDSCs from bladder cancer patients are suppressive, MDSCs were isolated by positively selecting CD15+ cells from urine samples. T-cells were isolated from PBMC and stimulated with OKT-3 alone or in combination with MDSCs for 72 hours. Supernatants were collected and IFN-gamma levels were analyzed by ELISA.

(4) Gemcitabine Treatment and ACT with OT-I:

To evaluate whether targeting MDSCs with gemcitabine (GEM) improves transfer of T cells, C57BL/6 mice received intravesical instillation of MB49-OVA tumor cells and were monitored via ultrasound. Seven-ten days after intravesical instillation of tumor, mice were separated into three treatment groups and an untreated (UNTX) group. The gemcitabine group (GEM) and the combination gemcitabine and OT-I treatment group (GEM+OT-I) received intravesical instillation of 500 ug gemcitabine for 30 minutes. The OT-I treatment group (OT-I) and GEM+OT-I treatment groups received intravesical instillation of 5×10⁶ OT-I T-cells for 3 hours at day 4 after instillation of Gem. Tumor growth was measured via ultrasound.

### b) Results:

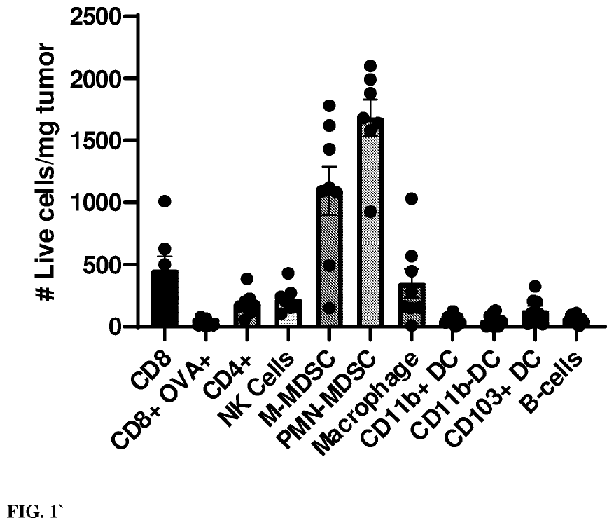

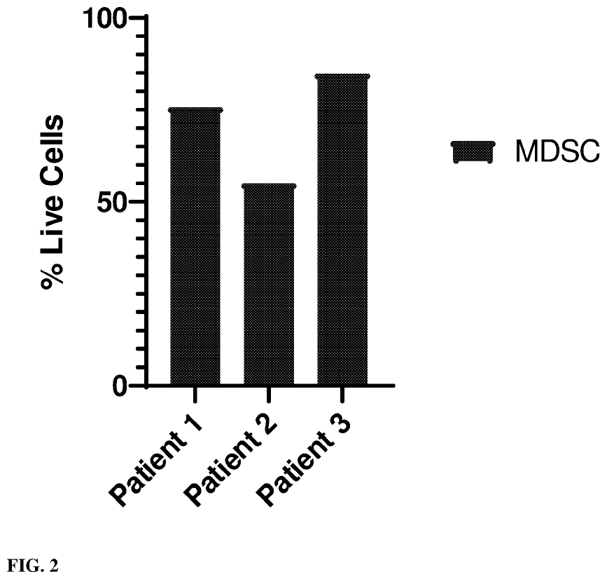

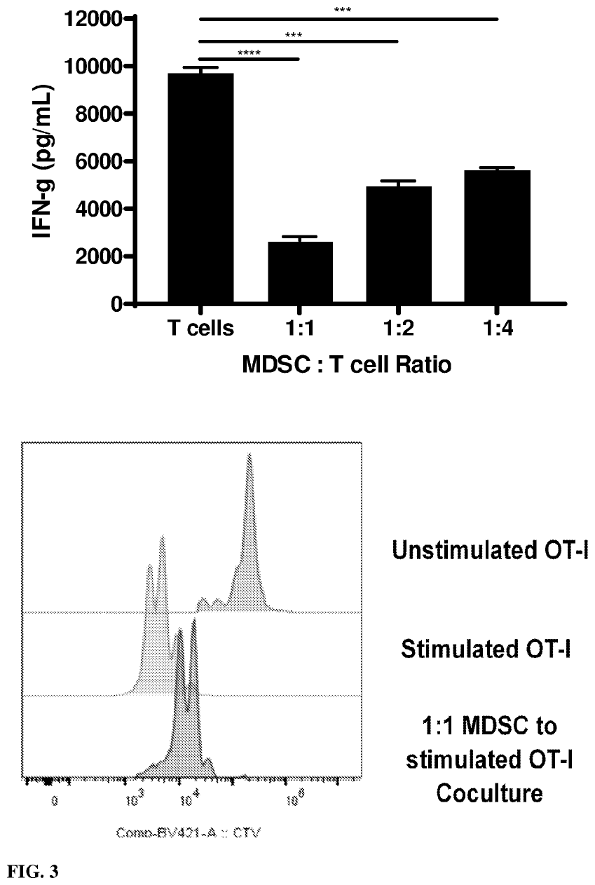

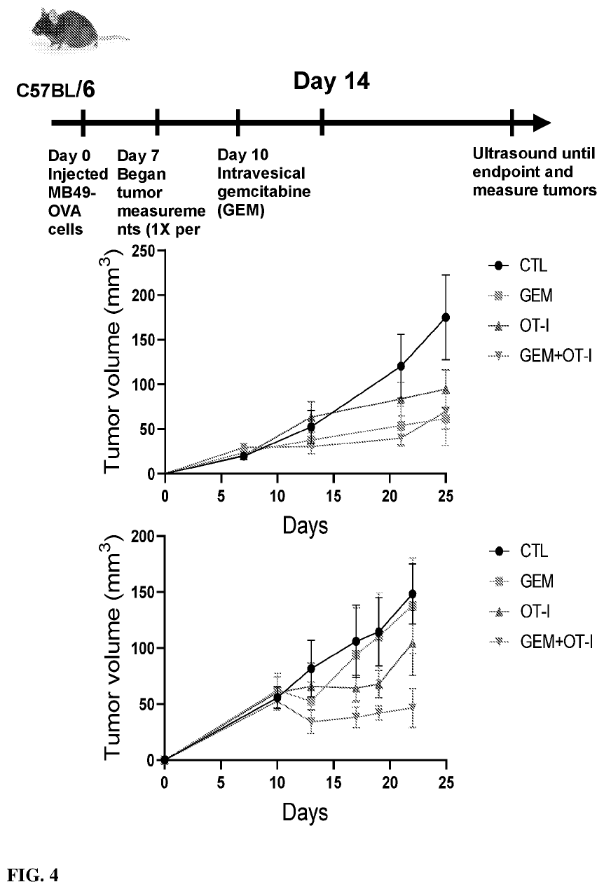

As shown in FIG. 1, bladder tumors are enriched for myelin derived suppressor cells (MDSCs). In mice bearing MB49-OVA tumors, the levels of polymorphonuclear (PMN)-MDSCs averaged 23.5% of live cells and monocytic (M)-MDSCs averaged 7.6% of live cells, demonstrating that nearly a third of live cells within murine bladder tumors are MDSCs. In the urine of bladder cancer patients, PMN-MDSCs predominantly make up the live cell population, averaging 71.7%, demonstrating an enrichment for MDSCs within the microenvironment of human bladder cancer (FIG. 2). MDSCs that infiltrate the tumor are suppressive. In murine coculture assays, MDSCs reduced the proliferation of OT-I T-cells and in human cocultures, MDSCs reduced T-cell IFN-gamma production to a fourth of control levels. Therefore, MDSCs. from bladder tumors suppress anti-tumor T-cells by inhibiting proliferation and reactivity (FIG. 3). In mice bearing large MB49-OVA tumors (>50 mm3), pretreatment with gemcitabine improved anti-tumor response in combination with ACT with OT-I T cells (FIG. 4). In smaller MB49-OVA tumors (<50 mm3), gemcitabine provided little added benefit to ACT.

### c) Conclusion:

MDSCs make up a significant proportion of the immune population within bladder tumors and exert suppressive effects on T-cells. Our studies support the selective depletion of MDSCs via gemcitabine to improve the anti-tumor effects of ACT.

1. A method of treating a cancer in a subject comprising administering to the subject a therapeutically effective amount of a nucleoside analog followed by the administration of a therapeutically effective amount of an adoptive cell therapy.

2. The method of treating a cancer of claim 1, wherein the nucleoside analog comprises gemcitabine, cytarabine, emtricitabine, lamivudine, zalcitabine, didanosine, vidarabine, abacavir, acyclovir, entecavir, stavudine, telbivudine, zidovudine, idoxuridine, or trifluridine.

3. The method of treating a cancer of claim 1, wherein the adoptive cell therapy comprises administering to the subject a therapeutically effective amount of tumor reactive immune cells.

4. The method of treating a cancer of claim 3, wherein the immune cells comprise chimeric antigen receptor (CAR) T cells, CAR natural killer (NK) (CAR NK) cells, tumor infiltrating lymphocytes, and/or marrow infiltrating lymphocytes.

5. The method of treating a cancer of claim 3, wherein the immune cells comprise T cells, natural killer (NK) cells, and/or NK T cells.

6. The method of treating a cancer of claim 1, wherein the cancer comprises bladder cancer.

7. A method of reducing the suppressive effects of myelin derived suppressor cells (MDSCs) in a subject receiving adoptive cell therapy for treatment of a cancer comprising administering to the subject a therapeutically effective amount of a nucleoside analog prior to administration of the adoptive cell therapy.

8. A method of increasing the efficacy of adoptive cell therapy (ACT) in a subject with a cancer comprising administering to a subject in need thereof a therapeutically effective amount of a nucleoside analog prior to administration of the adoptive cell therapy.

9. The method of reducing the suppressive effects of MDSCs of claim 7, wherein the nucleoside analog comprises gemcitabine, cytarabine, emtricitabine, lamivudine, zalcitabine, didanosine, vidarabine, abacavir, acyclovir, entecavir, stavudine, telbivudine, zidovudine, idoxuridine, or trifluridine.

10. The method of reducing the suppressive effects of MDSCs claim 7, wherein the cancer comprises bladder cancer.

11. The method of reducing the suppressive effects of MDSCs claim 7, wherein the adoptive cell therapy comprises administering to the subject a therapeutically effective amount of tumor reactive immune cells.

12. The method of reducing the suppressive effects of MDSCs of claim 11, wherein the immune cells comprise chimeric antigen receptor (CAR) T cells, CAR natural killer (NK) (CAR NK) cells, tumor infiltrating lymphocytes, and/or marrow infiltrating lymphocytes.

13. The method of reducing the suppressive effects of MDSCs of claim 11, wherein the immune cells comprise T cells, natural killer (NK) cells, and/or NK T cells.

14. The method of increasing the efficacy of ACT of claim 8, wherein the nucleoside analog comprises gemcitabine, cytarabine, emtricitabine, lamivudine, zalcitabine, didanosine, vidarabine, abacavir, acyclovir, entecavir, stavudine, telbivudine, zidovudine, idoxuridine, or trifluridine.

15. The method of increasing the efficacy of ACT of claim 8, wherein the cancer comprises bladder cancer.

16. The method of increasing the efficacy of ACT of claim 8, wherein the adoptive cell therapy comprises administering to the subject a therapeutically effective amount of tumor reactive immune cells.

17. The method of increasing the efficacy of ACT of claim 16, wherein the immune cells comprise chimeric antigen receptor (CAR) T cells, CAR natural killer (NK) (CAR NK) cells, tumor infiltrating lymphocytes, and/or marrow infiltrating lymphocytes.

18. The method of increasing the efficacy of ACT of claim 16, wherein the immune cells comprise T cells, natural killer (NK) cells, and/or NK T cells.
